(19) United States
(12) Patent Application Publication
(10) Pub. No.: US 2017/0349548 A1
(43) Pub. Date: Dec. 7, 2017

(54) P21-ACTIVATED KINASE INHIBITOR

(71) Applicant: Biosystem Consulting Limited Company, Ginowan-shi (JP)

(72) Inventors: Shinkichi TAWATA, Okinawa (JP); Cao Quan Binh NGUYEN, Kagoshima (JP); Nozomi TAIRA, Kagoshima (JP)

(73) Assignee: Biosystem Consulting Limited Company, Ginowan-shi (JP)

(21) Appl. No.: 15/539,947

(22) PCT Filed: Dec. 26, 2014
(86) PCT No.: PCT/JP2014/084514

§ 371 Date: Jun. 26, 2017

(51) Int. Cl. C07D 213/69; C07K 5/087; C07J 9/00; C07K 5/107; C07D 309/36

(52) U.S. Cl. CPC C07D 213/69; C07K 5/1016; C07D 309/36; C07J 9/00; C07K 5/0812

(57) ABSTRACT

The present invention addresses the problem of providing an inhibitor which has an excellent inhibitory activity on a p21-activated kinase. The present invention, by which has been solved the above-mentioned problem, is a p21-activated kinase 1 inhibitor containing, as an active ingredient, one or more compounds selected from the group consisting of dehydrokawain compounds, derivatives of dehydrokawain compounds, mimosine, derivatives of mimosine, and cucurbitacin compounds.

### TECHNICAL FIELD

The present invention relates to a p21-activated kinase 1 inhibitor, and more particularly it relates to a p21-activated kinase inhibitor which has an excellent inhibitory activity on a p21-activated kinase 1 participating in tumorigenesis and so on, and which contains as an active ingredient dehydrokawain compounds, mimosine, cucurbitacin compounds or their derivatives that are contained in tropical or subtropical plants.

### BACKGROUND ART

A p21-activated protein kinase (PAKs) family belongs to serine/threonine kinases depending on RAC/CDC42 and is grouped into 6 species (PAK1 to 6) in mammal. Among these species, PAK2 and PAK4 are essential for the development of embryo, but PAK1 is not essential for embryogenesis since a PAK1-defective mouse grows healthily and a PAK-defective variant of nematodes has a longer life than the wild type (NPLs 1 and 2).

On the other hand, it is known that PAK1 is essential for angiogenesis necessary in the proliferation or metastasis of solid tumor or in the growth of solid tumor. Excessive activation or excessive expression of PAK1 causes such a disease as cancer, type II diabetes, hypertension, Alzheimer's disease and so on (NPLs 1 and 2). Since PAK1 is not essential for the growth of normal cells, it does not cause any adverse effects even if inhibited because it is different from usual anti-cancer agents. Thus, a PAK1 inhibitor which is a selective low molecular compound is effective in therapy of a variety of PAK1-depending diseases or disorders. Although FRAX486 and FRAX596 are known so far to be the most potent inhibitors specific to PAK1, there are some problems that they are inferior in transcellular property, water solubility and bioavailability (NPL 3).

People in Okinawa have had in the longest healthy life in Asia. Some plants which are widely distributed in subtropical or tropical area including Okinawa such as white popinac, shell ginger, bitter melon, and the like, have been made it clear to have a variety of physiological activities, which have been believed to contribute to the health of Okinawa's people. It was so far unknown however that these plants had such a PAK1 inhibitory activity.

### CITATION LIST

### Non Patent Literature

NPL 1: Dummler B, Ohshiro K, Kumar R, Field J. Pak protein kinases and their role in cancer. Cancer Metastasis Rev. 2009; 28:51-63.
NPL 2: Maruta H. Herbal therapeutics that block the oncogenic kinase PAK1: A practical approach towards PAK1-dependent diseases and longevity. Phytother. Res. 2014; 28:656-672.
NPL 3: Dolan B M, Duron S G, Campbell D A, Vollrath B, Rao B S S, Ko H Y, Lin G G, Govindarajan A, Choi S Y, Tonegawa S. Rescue of fragile X syndrome phenotypes in Fmr1 KO mice by the small-molecule PAK inhibitor FRAX486. PNAS. 2013; 110:5671-5676.

### SUMMARY OF INVENTION

### Technical Problem

The purpose of the invention is to provide an inhibitor having an excellent inhibition activity on p21-activated kinase 1 (PAK1).

### Solution to Problem

As a result of intensive investigation to solve the above-mentioned problems, the present inventors have found that the specific compounds and their derivatives contained in tropical and subtropical plants such as shell ginger, white popinac (lead tree), bitter melon and the like, exhibit an excellent inhibitory activity on PAK1, thereby completing the present invention.

Thus the present invention provides a p21-activated kinase 1 inhibitor containing, as active ingredients, one or more compounds selected from the group consisting of dehydrokawain compounds, derivatives of dehydrokawain compounds, mimosine, derivatives of mimosine and cucurbitacin compounds.

### Advantageous Effects of Invention

The inhibitor of the invention shows an excellent inhibitory activity on p21-activated kinase 1 (PAK1) and has a high bioavailability with water solubility. Thus the inhibitor has an excellent therapeutic and prophylactic effect against PAK1-relating cancers as well as type II diabetes, hypertension, Alzheimer's disease, dementia and so on. In addition, it shows an excellent effect as anti-tumor agent since PAK1 participates in the growth or metastasis of solid tumors or in angiogenesis. In addition, since PAK1 is not essential for normal cells and its inhibition is hardly accompanied by adverse effect, the inhibitor of the invention is highly safe.

### BRIEF DESCRIPTION OF DRAWINGS

FIG. 1 shows a reaction scheme of mimosine tetrapeptide.

### DESCRIPTION OF EMBODIMENTS

In this invention, one or more compounds selected from the group consisting of dehydrokawain compounds and derivatives thereof, mimosine and derivatives thereof, and cucurbitacin compounds are used as active ingredients.

### (Dehydrokawain Compounds and Derivatives Thereof)

Dehydrokawain compounds are exemplified by those represented by the following formula (1):

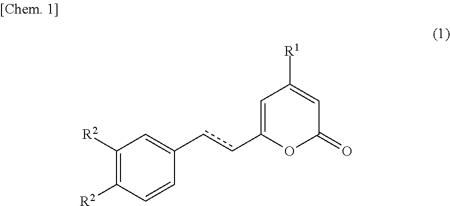

Wherein R¹ represents a hydroxyl group or methoxy group, and R² represents a hydroxyl group, methoxy group or hydrogen atom. The dotted line indicates the presence of absence of a bond.

As the compounds of the formula (1) used in the invention, 5,6-dehydrokawain (hereinafter sometimes referred to as “DK”) represented by the following formula (1a) and dihydro-5,6-dehydrokawain (hereinafter sometimes referred to as “DDK”) represented by the following formula (1b) can be cited.

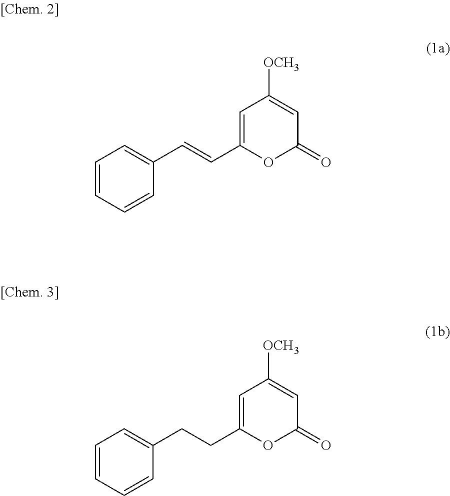 [SMILES: COc1cc(/C=C/c2ccccc2)oc(=O)c1 ; COc1cc(CCc2ccccc2)oc(=O)c1]

As for the derivatives of dehydrokawain compounds, the 5,6-dehydrokawain metabolites, that is, hispidin (6-(3,4-dihydroxystyryl)-4-methoxy-2H-pyran-2-one; (1c)) and derivatives thereof: H1 (6-(3,4-dimethoxystyryl)-4-methoxy-2H-pyran-2-one; (1d)), H2 (6-(3,4-dimethoxyphenethyl)-4-methoxy-2H-pyran-2-one; (1e)), and H3 (6-(3,4-dihydroxyphenethyl)-4-methoxy-2H-pyran-2-one; (1f)) can be exemplified.

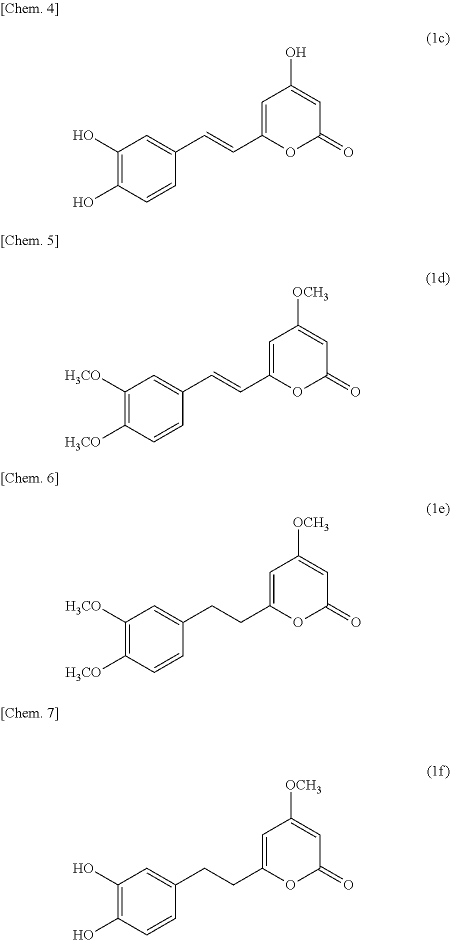 [SMILES: O=c1cc(O)cc(/C=C/c2ccc(O)c(O)c2)o1 ; COc1cc(/C=C/c2ccc(OC)c(OC)c2)oc(=O)c1 ; COc1cc(CCc2ccc(OC)c(OC)c2)oc(=O)c1 ; COc1cc(CCc2ccc(O)c(O)c2)oc(=O)c1]

Among these dehydrokawain compounds or derivatives thereof, hispidin and derivatives thereof H1-H3 show particularly excellent PAK1-inhibitory activities and so can be used preferably.

The above-mentioned DK and DDK can be isolated and purified, for example, from the extract of shell ginger according to the following manner.

Shell ginger (Gettou; Alpinia zerumbet), that is a perennial herb belonging to Alpinia (Gingiberaceae), is distributed in tropical or subtropical areas in Asia and in the Okinawa Prefecture or the southern part of Kyusyu in Japan. Although shell ginger may be used in any part of the 6 tissues (root, stem, leaf, flower, pericarp, and seed), it is appropriate to use the root as raw material for extraction. The material for extraction is preferably air-dried, then cut finely or ground into an appropriate size, and then used in the subsequent extraction step.

In the extraction step, an extracting solvent is added to the material for extraction provided as mentioned above in an amount corresponding to 5-100 parts by mass, and the extraction is carried out for a period of approximately from 20 minutes to 24 hours. The preferred extracting solvent used in the extraction includes, water, a lower alcohol such as ethanol, acetone, ethyl acetate, and the like, or a mixture of these solvents (hereinafter sometimes referred to as “aqueous solvent”). Among the above-mentioned aqueous solvents, the mixture may be a mixed solvent of ethanol and water in an optional ratio of about 10-96%. The extraction may be carried out preferably at a 6S temperature of about 50-100° C., during which operation the mixture may be stirred continuously or intermittently if required. The thus resulting extract of shell ginger may be subjected to gradient elution using column chromatography and so on to isolate and purify DK and DDK. DK and DDK may also be prepared by means of organic syntheses.

On the other hand, hispidin may be obtained from DK, for example, by conversion with a microsomal enzyme CYP2C9 of the rabbit's liver. In addition, the hispidin derivative H1 may be prepared from hispidin, for example, by the reaction with a well-known alkylating agent, for example, diazomethane. H2 may be prepared from H1 by hydrogenation with such a catalyst as palladium-carbon. H3 may also be prepared similarly from hispidin by hydrogenation. The reaction scheme is shown as follows.

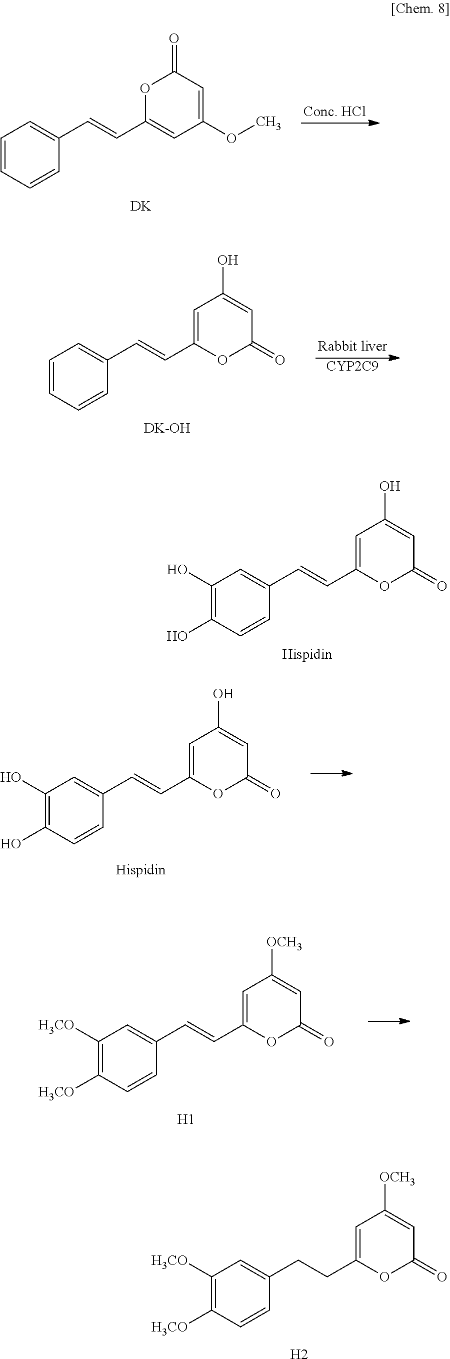

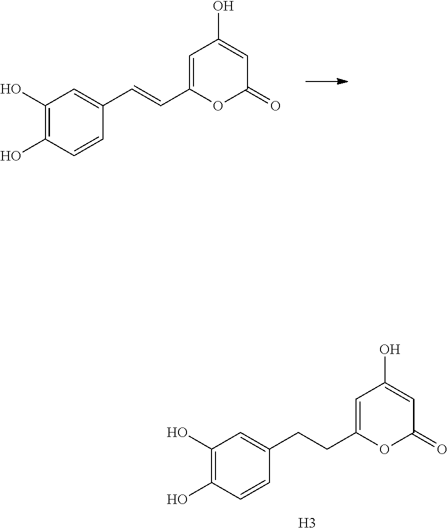 [SMILES: O=c1cc(O)cc(/C=C/c2ccc(O)c(O)c2)o1 ; O=c1cc(O)cc(CCc2ccc(O)c(O)c2)o1 ; "H3"]

### (Mimosine and Derivatives Thereof)

Mimosine (β-[N-(3-hydroxy-4-pyridone)]-α-aminopropionic acid) is a non-protein amino acid which has an alanine side-chain binding to the nitrogen atom of the pyridine ring (the following formula (2a)). The mimosine derivatives are exemplified by mimosinol represented by the following formula (2b) and mimosine tetrapeptide represented by the following formula (2).

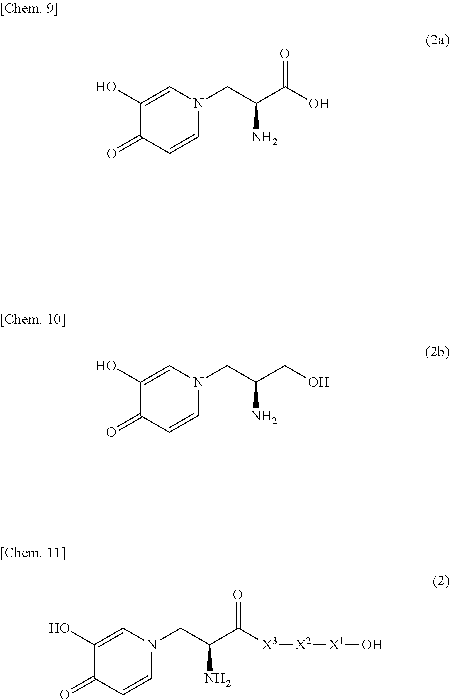

The mimosine derivatives of the above-mentioned formula (2) are tetrapeptides in which a tripeptide is bound to mimosine.

In the above-mentioned formula (2), there is no particular limitation in X¹-X³ as long as they are amino acid residues, and they are exemplified for example by alanine (Ala; A), arginine (Arg; R), asparagine (Asn; N), aspartic acid (Asp; D), cysteine (Cys; C), glutamine (Gln; Q), glutamic acid (Glu; E), glycine (Gly; G), histidine (His; H), isoleucine (Ile; I), leucine (Leu; L), lysine (Lys; K), methionine (Met; M), phenylalanine (Phe; F), proline (Pro; P), serine (SER: S), threonine (Thr; T), tryptophan (Trp; W), tyrosine (Tyr; Y), valine (Val; V) and the like; these may be independently the same as or different from each other. Among them, tyrosine (Tyr; Y), tryptophan (Trp; W) and phenylalanine (Phe; F) are preferred because they give an excellent activity as PAK1 inhibitors.

When there is an optical isomer in the amino acids corresponding to the groups X¹ to X³, it may be a D-isomer or L-isomer though the L-isomer is preferred. X³ is bound at the N-terminal side to form an amide bonding with mimosine.

Among these groups the case where, the group X³-X²-X¹ is a tripeptide residue selected from the group consisting of Phe-Phe-Tyr (FFY), Phe-Tyr-Tyr (FYY) and Phe-Trp-Tyr (FWY) is preferred because it has an excellent PAK1 inhibitory activity. The mimosine tetrapeptides in which the group X³-X²-X¹ is Phe-Phe-Tyr (FFY), Phe-Tyr-Tyr (FYY) or Phe-Trp-Tyr (FWY) are represented by the following formulae (2c: MFFY, 2d: MFYY, and 2e: MFWY).

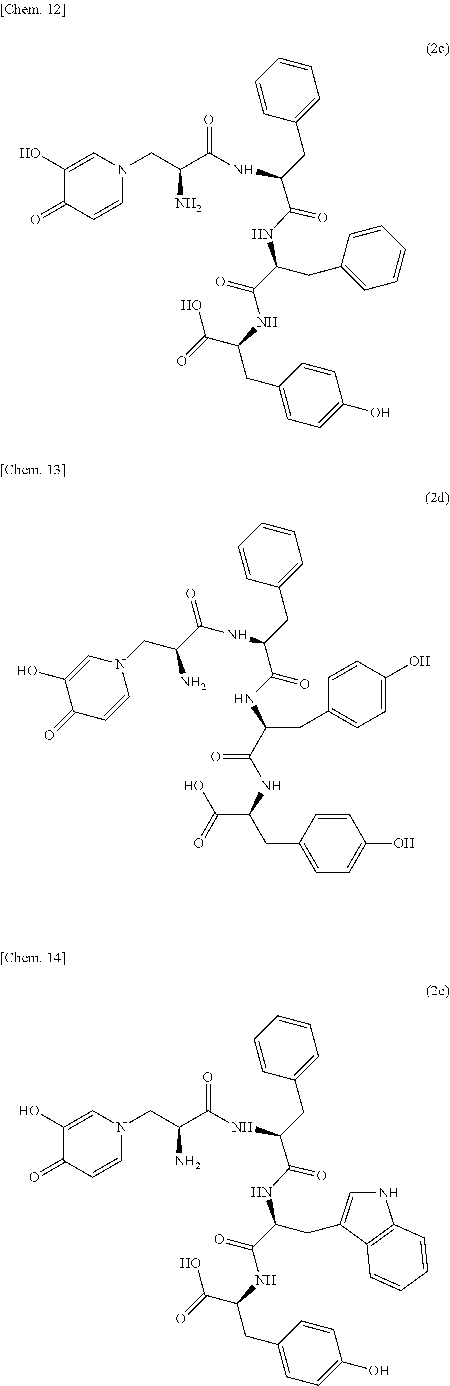 [SMILES: N[C@@H](Cn1ccc(=O)c(O)c1)C(=O)N[C@@H](Cc1ccccc1)C(=O)N[C@@H](Cc1ccccc1)C(=O)N[C@@H](Cc1ccc(O)cc1)C(=O)O ; N[C@@H](Cn1ccc(=O)c(O)c1)C(=O)N[C@@H](Cc1ccccc1)C(=O)N[C@@H](Cc1ccc(O)cc1)C(=O)N[C@@H](Cc1ccc(O)cc1)C(=O)O ; N[C@@H](Cn1ccc(=O)c(O)c1)C(=O)N[C@@H](Cc1ccccc1)C(=O)N[C@@H](Cc1c[nH]c2ccccc12)C(=O)N[C@@H](Cc1ccc(O)cc1)C(=O)O]

Further, the preferred mimosine derivatives used in the present invention may be represented by the following formula (2′)

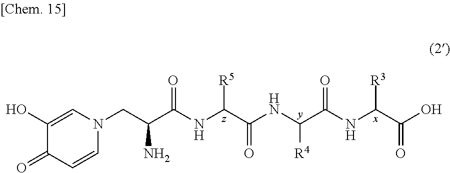

In the above-mentioned general formula (2), R³-R⁵ represents a group selected from the group consisting of a 4-hydroxybenzyl group, 3-indolylmethyl group and benzyl group; these groups may be independently the same as or different from each other.

In these groups, R³ is preferably a 4-hydroxybenzyl group. Additionally, R⁴ is preferably selected from the group consisting of a 4-hydroxybenzyl group, 3-indolylmethyl group and benzyl group. R⁵ is preferably a benzyl group.

In the above-mentioned formula (2), when the carbons to which are bounded R3, R4 and R5 are asymmetric ones, x, y and z each indicates a symbol of the absolute configuration (S or R). The mimosine derivatives used in the invention include their enantiomer, diastereomer and their mixture involving the racemate, though the (S)-configuration is preferred in all of the symbols x, y and z.

Mimosine, mimosinol, and mimosine tetrapeptides represented by the above-mentioned formula (2) or (2′) may be prepared for example according to the following method.

Mimosine is contained in the tropical and subtropical plants including white popinac or mimosa. White popinac is an evergreen shrub belonging to Leucaena, Mimosaceae, which is distributed in a tropical to subtropical area in Asia and in the Okinawa Prefecture to the southern part of Kyusyu in Japan.

In order to obtain mimosine from the leave of white popinac, the leave, preferably fresh and young leave, of white popinac are preferably cut finely or ground, and then used as raw materials for extraction.

Subsequently, the raw material for extraction provided as mentioned above is extracted with a proper quantity of hot water which has been heated in advance. The extraction may be conducted with a hot water at a temperature of 70° C. or higher, preferably 75° C. or nearly boiling state, but in order to deactivate mimosine-degradative-enzyme to yield highly pure mimosine, it is particularly preferable to use boiling water (approximately 100° C.) as hot water. The extraction is carried out for a period of 5 to 30 minutes, in particular for about 10 minutes preferably under boiling. As for the extraction solvent used in extraction, it is preferable to use distilled water, desirably with continuous or intermittent stirring if required during extraction.

Then, a strong positive ion-exchange resin is added to the extract of white popinac leave to make an component to be adsorbed containing mimosine adsorb. Then, the ion-exchange resin is washed with water or a mixture of water and ethanol, and then immersed in ammonia water and so on to elute mimosine from the ion-exchange resin. The resulting eluate is treated with active carbon if required, then concentrated, and allowed to stand at low temperature to yield the mimosine salt as precipitate, which is collected to obtain mimosine. The resulting mimosine may be purified by means of recrystallization and so on if required.

The thus resulting mimosine, as shown in the following reaction scheme, is allowed to react with tris(triethylsilyl)silyl triflate to yield mimosine tris(triethylsilyl)silyl ester (super silyl ester), which is then reduced with sodium borohydride and so on to yield mimosinol.

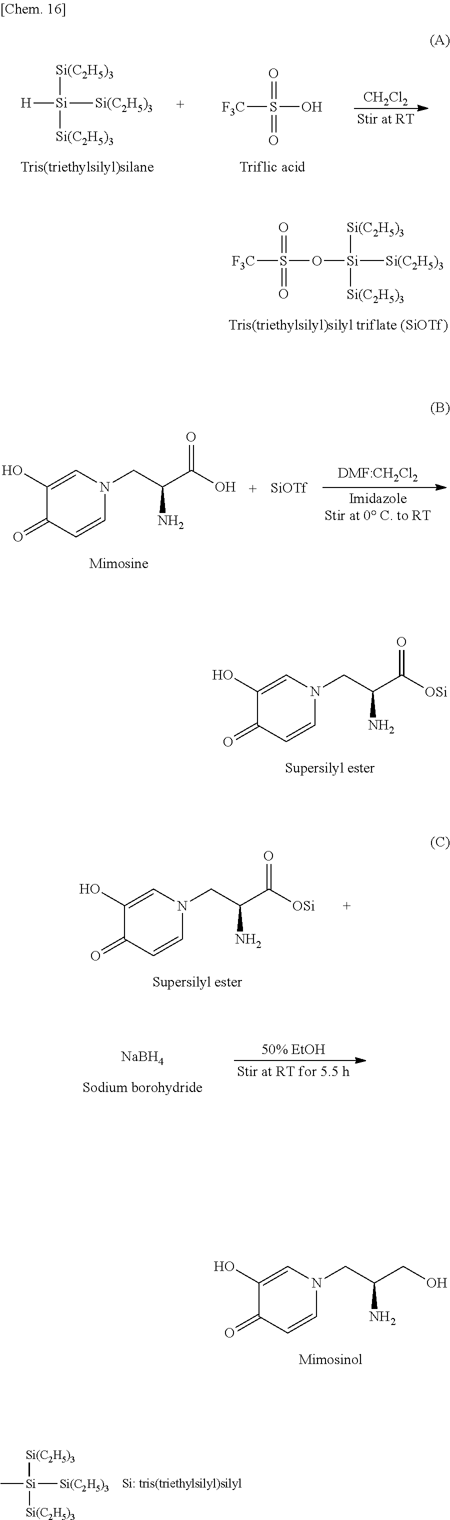

Mimosine may be bound to some amino acids to obtain the mimosine tetrapeptides by using a well-known method for peptide synthesis such as the solid-phase synthesis for peptides.

The amino groups of mimosine and amino acids are preferably protected with a protecting group or groups such as 9-fluorenylmethyloxycarbonyl group (Fmoc), t-butyloxycarbonyl group (Boc), and so on.

The condensing agent used for forming a peptide linkage includes, for example, diisopropylcarbodiimide (DIC), N,N-dicyclohexylcarbodiimide (DCC), and so on. These condensing agents may be used as a mixture with N-hydroxybenzotriazole (HOBt).

Boc and Fmoc, which are used as protecting groups for the amino-terminal amino group of the peptides or amino acids, may be removed with trifluoroacetic acid (TFA), piperidine, and so on.

As for the resin used in the solid-phase synthesis of peptides, the Wang resin and so on may be used. In detachment of the peptide from the resin for solid-phase synthesis, TFA may be used for example.

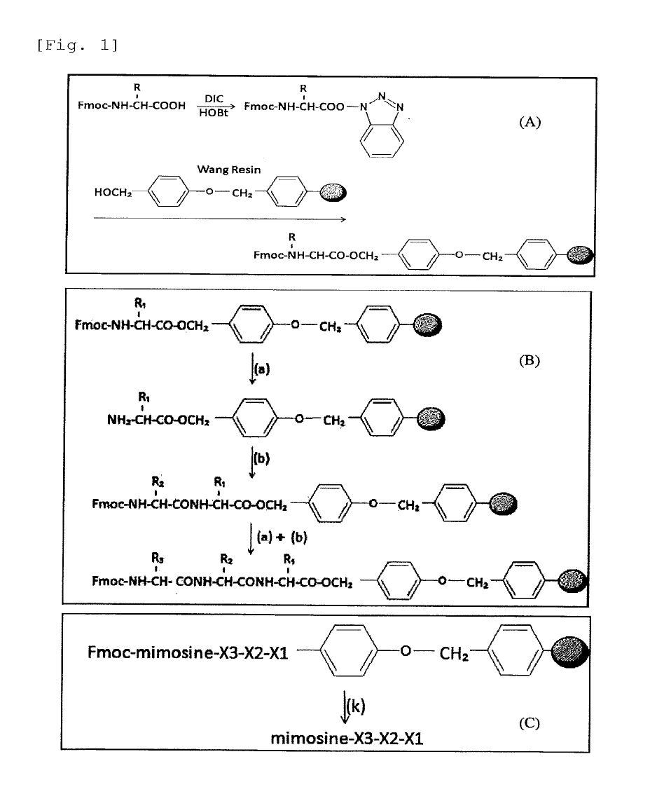

FIG. 1 shows the reaction scheme by the Fmoc solid-phase synthesis for producing mimosine derivatives used in the present invention. In this scheme, the Fmoc group of N-(9-fluorenylmethoxycarbonyloxy)succinimide (Fmoc-OSu) is bound to mimosine to yield Fmoc-mimosine, which is then bound to a tripeptide formed from Fmoc-amino acid to yield the mimosine tetrapeptide. The followings explain more specifically.

### (Preparation of Fmoc-Mimosine)

A solution of mimosine and sodium carbonate are dissolved in distilled water containing dioxane, to which is then added Fmoc-OSu, and the mixture is incubated at room temperature overnight. Then, a sodium carbonate solution is added, and after stirring the solution is filtered and then washed with ethyl acetate to remove the remaining unchanged Fmoc-OSu, and the by-products 9-fluorenylmethanol and 9-methylenefluorene. The aqueous fraction is adjusted to about pH 4 with hydrochloric acid in an ice bath to yield the crystals of Fmoc-mimosine as precipitate.

### (Solid-Phase Synthesis of Mimosine Tetrapeptide)

To a solution of Fmoc-amino acid (Fmoc-X₁—OH) in dimethylacetamide is added 1-hydroxy-1H-benzotriazole (HOBt) and N,N′-diisopropylcarbodiimide (DIC), and the mixture is stirred. To this solution is added the Wang resin which has been swelled in N,N-dimethylformamide (DMF), and the mixture is stirred (see FIG. 1A) The resin is filtered and washed with dichloromethane, isopropyl alcohol and methanol, and dried in vacuo. This is subjected to deprotection of Fmoc with 25% piperidine (reagent a) in DMF, and then the next amino acid (Fmoc-X₂—OH) is allowed to bind with the reagent b (a mixture of Fmoc amino acid, HOBt, HBTU and N,N-diisopropylethylamine (DIEA)) and further stirred (see FIG. 1B). In the same way this dipeptide is allowed to bind with Fmoc amino acid (Fmoc-X₃—OH) to yield a tripeptide. Further, Fmoc-mimosine prepared as mentioned above is allowed to bind in the same way and stirred with 95% trifluoroacetic acid (TFA; reagent k) (see FIG. 11C). The resin is filtered, washed with TFA, and the resulting filtrate is treated with ice-cooled diethyl ether to precipitate a mimosine tetrapeptide.

### (Cucurbitacin Compounds)

The cucurbitacin compounds include cucurbitacin A, B, C, D, E, F, G, H, I and so on, among which, compounds cucurbitacin I represented by the following formula (3) is preferably used since it has an excellent PAK1-inhibitory activity.

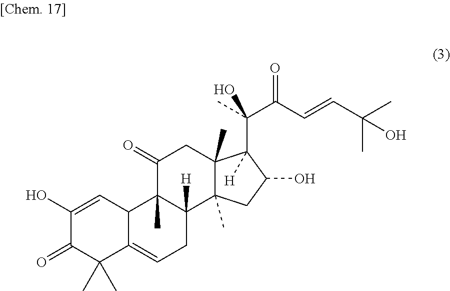

The cucurbitacin compounds can be isolated and purified from the plants of Cucurbitaceae such as bitter gourd (Momordica charantia), for example, as mentioned below.

Nigauri (bitter gourd) is called Goya (bitter melon) in Okinawa, of which the fruit part is used as food from which have been removed the seeds and pulp. Each part of the bitter melon is preferably air-dried, and cut finely or ground into an appropriate size to be used as raw material for extraction. The raw material for extraction, to which is added 1-20 parts by mass of extracting solvent, is extracted for a period of about 1-20 hours. As for the extracting solvent used in extraction, the above-mentioned aqueous solvent is preferably used. The preferred temperature for extraction is about 50-100° C. The thus resulting extract of bitter gourd is subjected to a well-known method for separation and purification such as column chromatography or preparative thin-layer chromatography to isolate the cucurbitacin compounds. In addition the products which are prepared by means of organic syntheses may be used.

The thus resulting dehydrokawain compounds and derivatives thereof, mimosine and derivatives thereof as well as cucurbitacin compounds may be used per se or if required after purification by a well-known method such as high performance liquid chromatography to be utilized as p21-activated kinase 1 (PAK1) inhibitor.

Alternatively, since the thus prepared shell ginger extract, white popinac extract, and bitter gourd extract contain such an active ingredient or ingredients as dehydrokawain compounds, mimosine and so on, these plant extracts may also be used as an active ingredient of p21-activated kinase 1 (PAK1) inhibitor in the invention.

In order to prepare a p21-activated kinase 1 (PAK1) inhibitor of the invention, a therapeutically effective amount of the above-mentioned active ingredient may be combined and mixed with a pharmaceutically acceptable optional ingredient or ingredients, for example, diluents, binders, lubricants, aqueous solvents, oily solvents, emulsifying agents, suspending agents, preservers, stabilizer, and so on.

The p21-activated kinase 1 (PAK1) inhibitor of the invention may be administered orally or parenterally. In case of oral administration, a conventional oral preparation in any one of the dosage forms, for example, solid preparations such as tablets, powders, granules, capsules and so on; solutions; oily suspensions; or liquid preparations including syrup or elixir, may be used. In case of parenteral administration, it may be used as aqueous or oily suspensions for injection, or as nasal drops.

The p21-activated kinase 1 (PAK1) inhibitor of the invention may usually be administered in case of oral administration at a dose of about 10-200 mg per day for an adult, preferably 10-50 mg, and more preferably about 10-20 mg, if required at divided doses of several times, though it is different depending on the active ingredient, the route of administration, the age, body weight and the condition of patient and the type of disease. In case of parenteral administration, it may be administered usually at a dose of 10-500 mg per day for an adult, preferably 10-20 mg, preferably about 5-10 mg.

The p21-activated kinase 1 (PAK1) inhibitor of the invention is able to cure, prevent or improve a disease or symptom in which PAK1 participates. Such a disease or symptom includes cancer, type-II diabetes, hypertension, Alzheimer's disease, dementia and so on. It can also be used as an anti-tumor agent because PAK1 participates in the growth or metastasis of solid tumor and in angiogenesis.

### EXAMPLES

The present invention will be explained more specifically by the following examples which are not intended to limit the invention.

### Preparation 1

### Preparation of DK and DDK:

Shell ginger (Alpinia zerumbet) was harvested on the campus at Okinawa University (1, Senbaru, Nishihara-cho, Nakagami-gun, Okinawa) To 2 kg of shell ginger was added 10 L of water, and the mixture was boiled for about 20 minutes. The extract was cooled at room temperature and filtered under suction (made by AS ONE Co., Shaking Baths SB-20). The filtrate was concentrated to 1 L in vacuo at 40° C., and extracted with hexane (3×500 mL). The organic layer was evaporated to dryness in vacuo. The crude extract after drying was boiled in water and filtered. The residue was separated and purified on HPLC to give DK. The filtrate was cooled to 4° C. to yield crystals, which were separated and purified by HPLC to give DDK. In the purification of DK and DDK, gradient elution was employed using 0.1% aqueous acetic acid solution (Solvent A) and 0.1% acetic acid-methanol solution (Solvent B) as a mobile phase. The gradient elution was conducted in the condition of congruent elution using a 1:1-mixture of Solvent A and Solvent B for the beginning 1-10 minutes, then according to the linear gradient in which Solvent B is changed from 50% to 100% for 10-20 minutes, then according to the congruent elution using 100% Solvent B for 20-30 minutes, and then accordingly the linear gradient in which Solvent B is changed from 100% to 50% for 30-35 minutes. The flow rate was 0.8 ml/min, and the wavelength of absorbance was at 280 nm.

### Preparation 2

### Preparation of Hispidin Derivatives (H1-3)

Hispidin (3 mg) was dissolved in 0.6 ml of a mixture of methanol and CH₂Cl₂ (1:5). This solution was cooled to 0° C., to which was added 0.5 ml of diazomethane/CH₂Cl₂ solution. The mixture was allowed to stand at 4° C. overnight. Solvent was distilled off, and the residue was purified by PTLC to give light yellow powder (2 mg, 67% yield). The compound H1 (3.5 mg) was dissolved in 0.82 mL of a mixture of MeOH CH₂Cl₂ (1:1) and stirred for 2 hours in the presence of 10% Pd/C (0.65 mg). The mixture was filtered, and the solvent was evaporated in vacuo. Purification by column chromatography gave the compound H2 as white solid (3 mg, 85%). In the same manner, H3 was prepared from hispidin. The followings indicate the ¹H-spectrum data of the resulting hispidin derivatives. In this connection, the data were recorded by JEOL JNM-ECA400 (JEOL, Japan). The chemical shift was indicated by ppm (δ) relating to TMS.

### (Hispidin Derivative H1)

¹H NMR (CDCl₃, 400 MHz) δ:

7.43 (d, 1H, CH), 7.07 (dd, 1H, CH), 7.00 (d, 1H, CH), 6.85 (d, 1H, CH), 6.43 (d, 1H, CH), 5.89 (d, 1H, CH), 5.46 (d, 1H, CH), 3.91 (s, 3H, OCH₃), 3.89 (s, 3H, OCH₃), 3.81 (s, 3H, OCH₃).

### (Hispidin Derivative H2)

¹H NMR (CDCl₃, 400 MHz) δ:

6.7 7 (d, 1H, CH), 6.69 (dd, 1H, CH), 6.66 (d, 1H, CH), 5.69 (d, 1H, CH), 5.40 (d, 1H, CH), 3.84 (s, 3H, OCH₃), 3.83 (s, 3H, OCH₃), 3.76 (s, 3H, OCH₃), 2.91 (m, 2H, CH2), 2.71 (m, 2H, CH2).

### (Hispidin Derivative H3)

¹H NMR (DMSO, 400 MHz) δ:

7.29 (d, 1H, CH), 7.20 (dd, 1H, CH), 6.76 (d, 1H, CH), 6.11 (d, 1H, CH), 5.26 (d, 1H, CH), 3.34 (m, 2H, CH2), 2.99 (m, 2H, CH2).

### Preparation 3

### Preparation of Mimosine:

The leave of white popinac (1.5 kg) harvested around the Department of Agriculture, Okinawa University, were boiled in 5 L of water for 10 minutes. After cooling, the extract was filtered under suction (made by AS ONE Co.; Shaking Baths SB-20), and 2 kg of an ion-exchange resin (Amberlite IRI 20 Plus (H)) was added to the filtrate. The mixture of the extract and the resin was stirred for 30 minutes and allowed to stand overnight. The ion-exchange resin was rinsed with distilled water 5-6 times, to which was dropwise added 5 L of 80% ethanol to eliminate chlorophyll. The resin was eluted with 6 L of 2N-ammonium hydroxide to give crude mimosine. This eluate was concentrated at 40° C. under reduced pressure to 300 mL, adjusted to pH 4.5-5.0 with 6N-hydrochloric acid, and kept in a refrigerator overnight to yield crystals. The resulting crystals were adjusted to pH 9.0 with 5N—NaOH, to which was added 6N—HCl to make pH 4.5-5.0 for recrystallization, and allowed to stand at 4° C. to give purified mimosine. Mimosine was kept at −20° C.

### Preparation 4

### Preparation of Mimosinol:

In a 25 ml round-bottom flask was placed 3.4 mL of solution of trifluoromethylsulfonic acid (187 μL, 2 mmol) in CH₂Cl₂, and the solution was stirred at room temperature. Then, a solution of tris (triethylsilyl) silane (618 μL, 2 mmol) was dropwise added, and the mixture was stirred at room temperature for 3 hours until the mixture became a clear solution. Mimosine (0.4 g, 2 mmol) was placed in the above-mentioned round-bottom flask, and then a mixture of imidazole (0.15 g, 2.2 mmol) contained in 3.4 mL of DMF-CH₂Cl₂ (1:1) was added. The reaction flask was cooled to 0° C., into which tris(triethylsilyl)silyl triflate was dropwise added. After termination of the dropwise addition, the reaction mixture was stirred at room temperature for 2 hours, and filtered. The solvent was distilled off from the filtrate to yield mimosine tris(triethylsilyl)silyl ester (hereinafter sometimes referred to as “Mimosine Ester”).

To 3 mL of 50% ethanol solution containing sodium borohydride (NaBH₄; 0.28 g, 7.2 mmol) was added 3 mL of 50% ethanol solution containing Mimosine Ester. The mixture resulting at room temperature was refluxed for 5.5 hours, and then the solvent ethanol was distilled off under reduced pressure. The resulting aqueous solution was extracted with ethyl acetate (3×20 mL), and the combined extracts were washed with a saturated sodium chloride solution, then dried on anhydrous sodium sulfate, and concentrated to yield 352 mg (95% yield) of mimosinol as colorless crystals. The followings indicate the ¹H-spectrum data of the resulting mimosinol.

### (Mimosinol)

1H NMR (D₂O, 400 MHz) δ:

7.63 (s, 1H, CH), 7.28 (s, 1H, CH), 5.64 (d, 2H, CH), 3.72 (d, 2H, CH2), 3.47 (d, 2H, CH2), 3.02-2.86 (m, 2H, CH).

### Preparation 5

### Preparation of Mimosine Derivative (MFFY)

The tetrapeptide was synthesized by binding a tripeptide to mimosine (M) by means of the Fmoc solid-phase synthetic method. The tripeptide was prepared by using the Fmoc-amino acid obtained from the HiPep Laboratories through the first step of binding with tyrosine (Y), then binding with phenylalanine (F) forming the dipeptide, and then further with phenylalanine (F) forming the tripeptide. The thus resulting dipeptide was bound to the separately prepared Fmoc-mimosine to yield the mimosine tetrapeptide. The followings show a more specific method for the preparation.

### (Preparation of Fmoc-Mimosine)

To a solution of 5 g of mimosine and 5.5 g of sodium carbonate dissolved in 75 mL of distilled water containing 75 mL of dioxane was added 12.5 g of N-(9-fluorenylmethoxycarbonyloxy)succinimide (Fmoc-OSu), and the mixture was incubated at room temperature overnight. Then, 300 mL of sodium carbonate solution (0.1M) was added, and the mixture was further stirred by a magnetic stirrer (300 rpm) for 5 hours at 25° C. The resulting solution (450 mL) was filtered and washed with ethyl acetate (150 mL) to remove the Fmoc-OSu remaining unchanged and the by-products 9-fluorenylmethanol and 9-methylenefluorene. The aqueous fraction was decreased to pH 4 with 6N-hydrochloric acid in an ice bath to yield Fmoc-mimosine as crystals. This was collected by filtration and dried in vacuo (yield: 7.108 g).

### (Solid-Phase Synthesis of Mimosine Tetrapeptide)

To 5 mL of dimethylacetamide solution of 1.6 mmol of Fmoc-L-tyrosine were added 1.6 mmol of 1-hydroxy-1H-benzotriazole (HOBt) and 1.6 mmol of N,N′-diisopropylcarbodiimide (DIC), and the mixture was stirred for 10 minutes. To this solution was added 1 g of Wang resin expanded in DMF, and the reaction mixture was stirred for 17 hours (see FIG. 1A). The resin was collected by filtration, washed with dichloromethane, isoproopyl alcohol and methanol, and dried in vacuo. After deprotection of Fmoc with 25% piperidine (reagent a) in DMF for 30 minutes, the subsequent amino acid Fmoc-L-phenylalanine was combined to the resin mixture (Fmoc-amino acid:HOBt:HBTU:N,N-diisopropylethylamine (DIEA)=4:3:3.6:8: reagent b). The reaction mixture was further stirred for 1 hour (see FIG. 1B).

In order to confirm the perfection of binding, a ninhydrin test was performed. A mixture of HOBt, acetic acid, DIEA, and DMF (0.8:19:9:400) was used at a rate of 20 mL for 1 g of the resine to protect the unbinding Fmoc-L-tryptophan with an acetyl group.

Subsequently, in the same manner as mentioned above, Fmoc-L-phenylalanine was combined to the dipeptide. Further, Fmoc-mimosine prepared as mentioned above was added and combined similarly and the resulting resin was slowly agitated in 95% trifluoroacetic acid (TFA; reagent k) for 1 hour (see FIG. 1C). After filtration, the resin was washed with TFA, and the resulting filtrate was treated with ice-cooled diethyl ether to yield precipitate. The resulting precipitate was collected by filtration, washed three times with diethyl ether, and dried in vacuo to yield the desired mimosine tetrapeptide (M-FFY). The crude peptide was obtained as white solid in yield of 80.2 mg. This crude peptide was further purified by liquid chromatography according to the following condition. LC-MS (ESI−) m/z: 693.2 ([M-H]⁺)

### (Condition of HPLC)

Column: Cadenza CD-C18 Column (20×100 mm; 3 μm)

Mobile phase: 0.1% trifluoroacetic acid/CH₃CN (1.5/8.5)

Flow rate: 5 mL/minute

### Preparation 6

### Preparation of Mimosine Derivative (MFYY):

Mimotine tetrapeptide (M-FYY) was prepared (in yield of 65.7 mg) in the same manner as in Preparation 1, except that Fmoc-L-tyrosine (Y) was used as the Fmoc amino acid to be combined secondarily, and Fmoc-L-phenylalanine (F) as Fmoc amino acid to be combined thirdly. LC-MS (ESI−) m/z: 670.1 ([M-H]⁺)

### Preparation 7

### Preparation of Mimosine Derivative (MFWY):

Mimotine tetrapeptide (M-FWY) was prepared (in yield of 71.5 mg) in the same manner as in Preparation 1, except that Fmoc-L-tryptophan (W) was used as the Fmoc amino acid to be combined secondarily, and Fmoc-L-phenylalanine (F) as Fmoc amino acid to be combined thirdly. LC-MS (ESI−) m/z: 654.2 ([M-H]⁺)

### Example 1

The PAK inhibitory activities of the dehydrokawain compounds, mimosine or derivatives thereof and cucurbitacin I were determined. Assay of the PAK1 inhibitory activity was carried out using an ADP-Glo™ kinase assay kit (V4479, Promega, Madison, Wis.). Human PAK1 (10 μL) at a reaction concentration of 25 ng was incubated with 5 μL of the test compounds at the respective concentrations for 10 minutes. At the beginning of the reaction, 2.5×ATP/substrate mix (10 μL) was added and incubated for 40 minutes. The reaction was terminated by adding L of ADP-Glo reagent. In addition, 50 μL of Kinase detection reagent was added to cause the conversion of ADP to ATP and the emission reaction of freshly synthesized ATP. After incubation for 30 minutes, the intensity of emission in each well was measured by a Microplate Reader (MTP-880Lab, Corona) for 0.5 seconds as integration time. To the blank well was added all of the ingredients except for the test compounds and enzymes. The procedure was conducted at room temperature all the time. The inhibition rate was determined as the value on the kinase activity of control to which the inhibitor was not added. Table 1 shows the IC₅₀ of each test compound. In this connection, the IC₅₀ for quercetin, resveratrol, and curcumin were also determined in the same manner.

TABLE 1

| Test Compound | IC₅₀ (μM) |
|---|---|
| Mimosine | 37 |
| Mimosinol | 30 |
| MFFY | 0.13 |
| MFYY | 2.3 |
| MFWY | 0.60 |
| DK | 17.1 |
| DDK | 10.3 |
| Hispidin | 5.7 |
| H1 | 1.6 |
| H2 | 1.2 |
| H3 | 2.0 |
| Cucurbitacin I | 19 |
| Quercetin | 340 |
| Resveratrol | 15 |
| Curcumin | 7.0 |

As shown in Table 1, all of the test compounds show the PAK1 inhibitory activity. IC₅₀ of mimosine and mimosinol were 37 and 30 μM respectively, while those of DK and DDK were 17 and 10 μM, indicating that DK and DDK have a significantly stronger PAK1 inhibitory activity than mimosine and mimosinol. In addition, the DK metabolite hispidin had a strong PAK1 inhibitory activity (IC₅₀=5.7 μM). This value is almost equivalent to that of curcumin (IC₅₀=7.0 μM), which is markedly stronger than that of resveratrol (IC₅₀=15 μM). From these results, it was suggested that the methoxy group at the C-5 position of DK and DDK possibly contributes to the PAK1 inhibitory activity.

It is considered that DK will act in vivo as a PAK1 inhibitor by itself or as hispidin after converted by an enzyme CYP2C9. The PAK1 inhibitory activity of DK is weaker than its metabolite hispidin. Accordingly, it might be highly possible that the two OH groups binding to the benzene ring of DK or DDK will contribute to enhancement of the PAK1 inhibitory activity.

The Hispidin derivatives H1 to H3, which were synthesized in order to enhance the PAK1 inhibitory activity, had a higher PAK1 inhibitory activity than hispidin in all cases.

The mimosine tetrapeptides had also high PAK1 inhibitory activities. In particular, MFFY and MFWY had IC₅₀'s of 0.13 and 0.60 μM, respectively, and inhibited PAK1 at a level of nanomolar concentration.

### INDUSTRIAL APPLICABILITY

The inhibitor of the present invention, since having an excellent PAK1 inhibitory activity, is available as a drug and the like for curing or preventing such a PAK1-relating disease as cancer or type II diabetes.

1: A p21-activated kinase inhibitor, comprising at least one selected from the group consisting of a dehydrokawain compound, a derivative of a dehydrokawain compound, mimosine, a derivative of mimosine and an cucurbitacin compound as an active ingredient,
wherein:
the dehydrokawain compounds and derivatives thereof are at least one selected from the group consisting of 5,6-dehydrokawain, dihydro-5,6-dehydrokawain, hispidin, 6-(3,4-dimethoxystyryl)-4-methoxy-2H-pyran-2-one, 6-(3,4-dimethoxyphenethyl)-4-methoxy-2H-pyran-2-one, and 6-(3,4-dihydroxyphenethyl)-4-methoxy-2H-pyran-2-one;
the mimosine and derivatives thereof are at least one selected from the group consisting of mimosine, mimosinol and a mimosine tetrapeptide represented by the following formula (2): 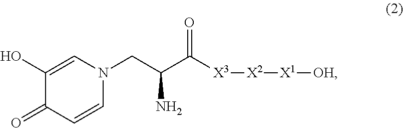
in which the group X³-X²-X¹ indicates a tripeptide residue selected from the group consisting of Phe-Phe-Tyr, Phe-Tyr-Tyr and Phe-Trp-Tyr; and
the cucurbitacin compound is cucurbitacin I.

2-6. (canceled)

7: An anti-tumor agent, comprising the p21-activated kinase inhibitor according to claim 1 as an active ingredient.

8: A prophylactic or therapeutic agent for diabetes, comprising the p21-activated kinase inhibitor according to claim 1 as an active ingredient.

9: A prophylactic or therapeutic agent for hypertension, comprising the p21-activated kinase inhibitor according to claim 1 as an active ingredient.

10: A prophylactic or therapeutic agent for Alzheimer's disease, comprising the p21-activated kinase inhibitor according to claim 1 as an active ingredient.
